(19) United States
(12) Patent Application Publication
(10) Pub. No.: US 2004/0128166 A1
(43) Pub. Date: Jul. 1, 2004

(54) Sales system and method for prescription products

(76) Inventors: Toshio Yamamoto, Setagaya-Ku (JP); Koju Nishi, Sakura-Shi (JP); Emi Takehana, Edogawa-Ku (JP); Tadashi Morigaki, Ichikawa City (JP); Randall B. Pugh, Jacksonville, FL (US); Christopher C. McMorrow, Jacksonville, FL (US); Evelyn R. Aguilar, Jacksonville, FL (US); Janet J. Plapp, Jacksonville, FL (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/681,974

(22) Filed: Oct. 9, 2003

Related U.S. Application Data
(60) Provisional application No. 60/419,402, filed on Oct. 18, 2002.

(51) Int. Cl. G06F 17/60

(52) U.S. Cl. 705/2

(57) ABSTRACT

The present invention relates to a system for ordering at least one prescribed product comprising: an authorized reseller system comprising an authorized reseller registration system wherein at least one prescription is entered; a consumer system comprising a consumer registration system and a prescribed product order placement system; and

a prescription management system comprising a prescription verification function which comprises performing at least one calculation on the at least one prescription entered by the reseller to generate a calculated approved prescription for the at least one product for the at least one patient wherein the calculated approved prescription includes a maximum purchase amount of prescribed product that may be purchased under the calculated approved prescription and an actual wear schedule wherein said authorized reseller system, said consumer system and said prescription management system are communicatively linked so that the consumer may order prescribed products directly from a manufacturer of prescribed product so long as the amount of prescribed products ordered does not exceed the maximum purchase amount or the actual wear schedule.

### RELATED PATENT APPLICATIONS

This patent application claims priority of a provisional application, U.S. Ser. No. 60/419,402 which was filed on Oct. 18, 2002.

### BACKGROUND OF INVENTION

1. Field of the Invention

The present invention is directed generally to a method and apparatus for managing information and data flow, and, more particularly, to an apparatus and method for medical based information and supply flow management.

2. Description of the Background

Historically, an individual seeking to obtain personal medical information and/or supplies related to a prescription, such as the fulfillment of a prescription for contact lenses, had few options available. Typically, personal medical information was available for a patient only at the point of care, such as of an eye care professional, or in the course of obtaining medical supplies, such as with the need of a prescription.

Due to the fact that access to personal medical information is generally available only at the point of care, patients or consumers desiring to access personal medical information generally must do so in person. Security and privacy concerns allow only limited access to such information by patients or consumers over the phone or over the internet, in part due to the fragmented and/or non-uniformly organized nature of independent healthcare professionals and their patient records.

As a result of restricting access to such medical information, patients often have difficulty obtaining paperwork needed, such as, for example, prescriptions for contact lenses, that would allow them to purchase necessary medical supplies, such as contact lenses and related supplies. Commonly, a patient needing certain medical supplies would first need to obtain necessary paperwork from the point of care, and subsequently present that paperwork to an authorized reseller from which medical supplies could be obtained. However, an authorized reseller acting on the request of the patient may fail in properly delivering the necessary medical supplies, due to the lack of communication of the patient's needs to the manufacturer of medical supplies. Additionally, any changes in the paperwork for the patient often require that the patient return to the point of care to obtain new paperwork, which new paperwork may only then be delivered to the authorized reseller to obtain the necessary medical supplies. Changes in paperwork can occur because of newfound medical needs, changes in those medical needs, or the expiration of dated medical supplies or paperwork, thereby creating a constant need for the consumer or patient to repeatedly return to the point of care professional.

Further, the lack of uniform record keeping, such as among a healthcare professional, manufacturer, and/or authorized reseller of medical supplies, increases the likelihood of breakdowns in communications between these necessary parties to properly get medical supplies to the patient. Such breakdowns also may cause inconvenience to the patient, or cause a patient to use incorrect medical supplies.

Thus the need exists for a medical based information and supplies flow management system that may be conveniently accessed from any communications device, irrespective of internet activity, that provides a central access point and data management point, and that improves medical information flow and supply flow between patient, consumer, authorized reseller, manufacturer, and point of care professional.

### A BRIEF DESCRIPTION OF SEVERAL VIEWS OF THE DRAWINGS

For the present invention to be clearly understood and readily practiced, the present invention will be described in conjunction with the following figures, wherein like reference numerals designate like elements, and wherein;

FIG. 1 is a block diagram illustrating the flow of medical based information and supply;

FIG. 2 is a flow diagram illustrating the registration of authorized reseller information as employed in FIG. 1;

FIG. 3 is a flow diagram illustrating the flow of patient medical information as employed in FIG. 1;

FIG. 4 is a flow diagram illustrating the entering of at least one prescription, the flow of at least one prescription, and the storage of information relating to at least one prescription;

FIG. 5 is a flow diagram illustrating the method of consumer registration as employed in FIG. 1;

FIG. 6 is a flow diagram illustrating the interaction between the consumer and the present invention;

FIG. 7 is a flow diagram illustrating the method of perscription maintenance;

### DETAILED DESCRIPTION OF THE INVENTION

It is to be understood that the figures and descriptions of the present invention have been simplified to illustrate elements that are relevant for clear understanding of the present invention, while eliminating, for purposes of clarity, many other elements found in a typical information flow system and method. Those of ordinary skill and art will recognize that other elements are desirable and/or required in order to implement the present invention. However, because such elements are well known in the art, and because they do not facilitate their understanding of the present invention, a discussion of such elements is not provided herein. Additionally, the following definitions are provided to aid in understanding the usage of the terms employed in this specification:

Manufacturer: The entity that produces, sells, and ships products and collects payment from Authorized Resellers. The Manufacturer may have a relationship with multiple Authorized Resellers.

Authorized Reseller: An entity that has a relationship with the Manufacturer, as well as with one or more Patients and which provides services and/or products to patients, consumers or doctors. The Authorized Reseller may be a doctor, an oculist, a retail Contact Lens shop, or other similar entity, for example.

Patient: The individual that uses products produced by the Manufacturer and prescribed by a doctor, an oculist, or other similar entity.

Consumer: The individual that uses the Consumer System to order products for a Patient. In most cases the Consumer will be the same individual as the Patient. In some cases, the Consumer may place orders on behalf of a Patient, such as when the Patient is a child and the Consumer is the parent.

Authorized Reseller System: A system, supplied and maintained by the Manufacturer, for use by the Authorized Resellers.

Consumer System: A system for use by Consumers associated with Authorized Resellers, which Authorized Resellers are using the Authorized Reseller System.

Oculist: Any person that can legally fit and prescribe an ophthalmic device.

Product: An opthalmic device or related good.

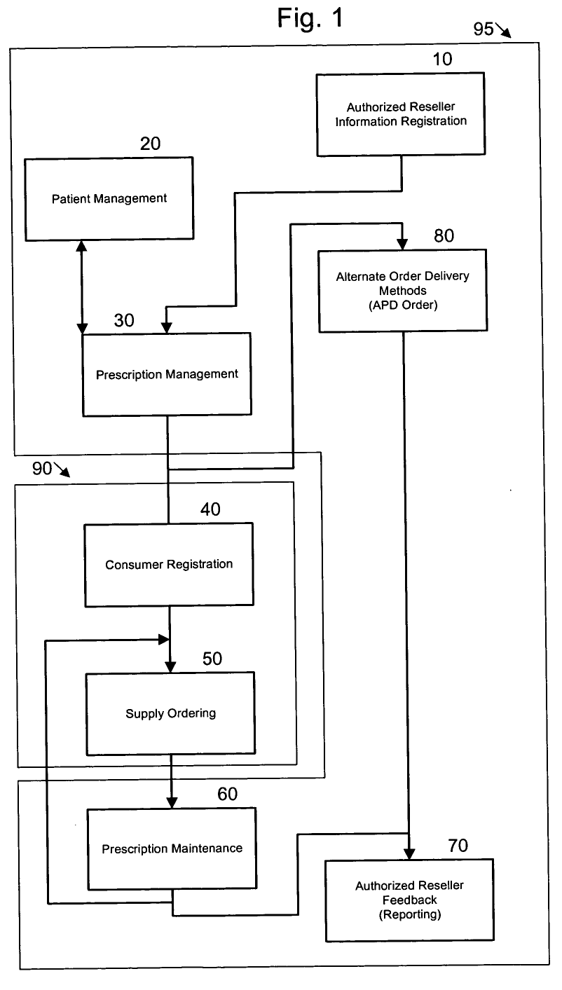

FIG. 1 is a block diagram illustrating an embodiment of the present invention. An authorized reseller information registration system 10 communicates with a patient management system 20 and a prescription management system 30, which system 30 provides access to the consumer system 90. The consumer system 90 supports a consumer registration system 40 that allows a patient access to an online ordering system 50. Using the authorized reseller system 95, the authorized reseller can place an order on behalf of a patient, the manufacturer processes the order and ships it to the patient's designated delivery address, the manufacturer bills the authorized reseller for the products shipped, and the authorized reseller collects payment based on the authorized reseller's retail price. These exchanges of payment may occur electronically, such as by online credit acounts or automated withdrawal, as will be apparent to those skilled in the art. Alternatively, a consumer may request registration to the consumer system 90 by an authorized reseller to allow that consumer to access prescription information from that authorized reseller online. Additionally, the consumer system 90 and the authorized reseller system 95 may provide information that supports the authorized reseller feedback reporting system 70. System 70 may include various types of reporting available to authorized resellers, such as for consumer service feedback. Reporting functions 70 include, but are not limited to, consumer sales reports, consumer service reports, order status reports and various financial reports.

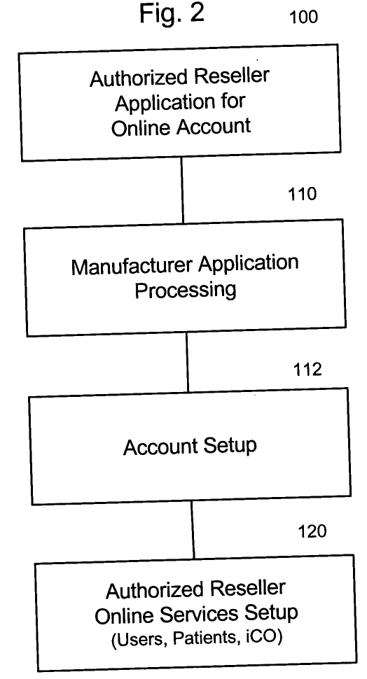

FIG. 2 is a flow diagram illustrating the authorized reseller information registration of authorized reseller system 95, wherein the processing of authorized reseller applications 110, the manufacturer initial account setup 112, and the set up of authorized reseller online services 120 occur. The authorized reseller registration 100 provides the manufacturer with a completed contract by the authorized reseller, requesting online access to the authorized reseller system 95. The initial account setup 112 enables an authorized reseller to access the authorized reseller system 95, and issues a user id and password for the authorized reseller. Upon qualification, the authorized reseller is able to use a communication device, such as personal computer or other network-capable device, to gain access through a network to the authorized reseller system 95, which system 95 may, for example, be provided by the manufacturer. The authorized reseller uses the user id and password provided by the initial account setup process 112 to gain authenticated access to the authorized reseller system 95. The authorized reseller enters account information into the authorized reseller online services setup 120 that may be displayed, used or accessed in the consumer system 90. Specific fields required within the authorized reseller online services setup 120 may vary, such as by country. Authorized reseller specific information collected and entered into the authorized reseller online services setup 120 may include, but is not limited to, authorized reseller name, address, telephone number, email address, website address, hours of operation, return policy, privacy policy, and insurance plan information. In addition, the authorized reseller selects a unique Authorized Reseller code for use in the consumer registration process 40, as described herein below.

In an embodiment, the authorized reseller information registration 10 may include administrative and ordering functions within the authorized reseller system 95 that can be provided in conjunction with, or independent of, functions provided to support the consumer system 90. These functions may include, but are not limited to, the ability to add, modify or delete user ids or adjust authorized reseller accounts, change the logged on user password, place stock orders to replenish office inventory, place orders for delivery to consumers, place orders for sales support and materials, check the status of orders that have been placed, and maintain patient records.

In order to enter prescriptions to allow consumers to place orders through the consumer system 90, the qualifying authorized reseller completes several set up steps in the authorized reseller online services setup 120. The steps described below can be completed in any order and entered into the authorized reseller system so that prescriptions can be entered.

The authorized reseller enters information, including, for example, prescription defaults, into the authorized reseller online services setup 120, which information may be used in various calculations and algorithms associated with patient prescriptions. Settings may include the default prescription duration and/or a rounding rule to be applied for calculations of suggested quantity 320 described herein below.

The authorized reseller enters product prices to be charged when the consumer uses the consumer system 90 to place an order. The prices entered by the authorized reseller may include a standard price and/or optional alternate (e.g. discounted) pricing for each of the manufacturers products that are available for sale through the authorized reseller.

The authorized reseller enters delivery prices to be charged when the consumer uses the consumer system 90 to place an order. The delivery methods and pricing structures may vary. Delivery prices may include a standard price and/or alternate (e.g. discounted) pricing for each available delivery method.

The authorized reseller must add at least one doctor, oculist, or other similar entity in authorized reseller online services setup 120 to be associated with prescriptions. The authorized reseller can add, modify and/or inactivate doctors, oculists, or other similar entities at any time. At least one doctor, oculist, or other similar entity must be active in order for prescriptions to be entered into the authorized reseller system 30. The specific details associated with each doctor, oculist, or other similar entity may vary, and may include, but are not limited to, the name, the practice name, practice address and any applicable insurance information.

After completion of the authorized reseller online services setup 120, the authorized reseller can optionally, at any time, enter or modify a sales campaign. A sales campaign has a beginning date, an ending date, and/or special pricing for one or more products. The special pricing may include, but is not limited to, a campaign base price and/or discounted pricing for any or all of manufacturer's products that are available for sale through the authorized reseller. Sales campaign pricing may be automatically displayed and/or applied to patient orders that contain products included in the campaign when those orders are placed during the specified sales campaign term.

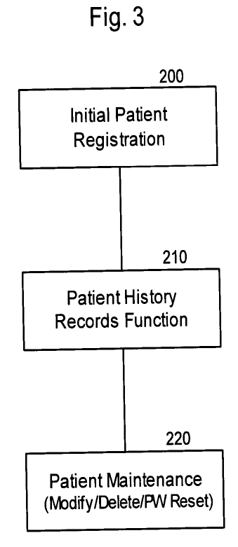

FIG. 3 is a flow diagram illustrating patient management 20 of the present invention. The patient management 20 of the present invention may include, for example, patient history records 210, patient maintenance 220 or the like. The patient management 20 provides the ability to add, edit and delete patients in the authorized reseller system 95 and/or the consumer system 90. Information collected during initial patient registration 200 includes, but is not limited to, the name, address, phone number, date of birth, and e-mail address of the patient. When a patient is added to the authorized reseller system, a unique patient number is assigned. In an embodiment of the invention, patient history records 210 can be associated with individual patients. Dated text descriptions entered by the authorized reseller can be used to track information and activity for a patient. Once entered, these records can be viewed, but preferably cannot be altered or deleted. Additionally, patient maintenance 220 allows the authorized reseller to manage the password for any consumer registered to use the consumer system 90. If the information in the patient maintenance 220 is modified, the authorized reseller system 95 may send a notification, such as an an e-mail, to the consumer, informing the consumer that information associated with that id has been changed. For example, the consumer may be notified that his/her password has been reset to a default known to the consumer.

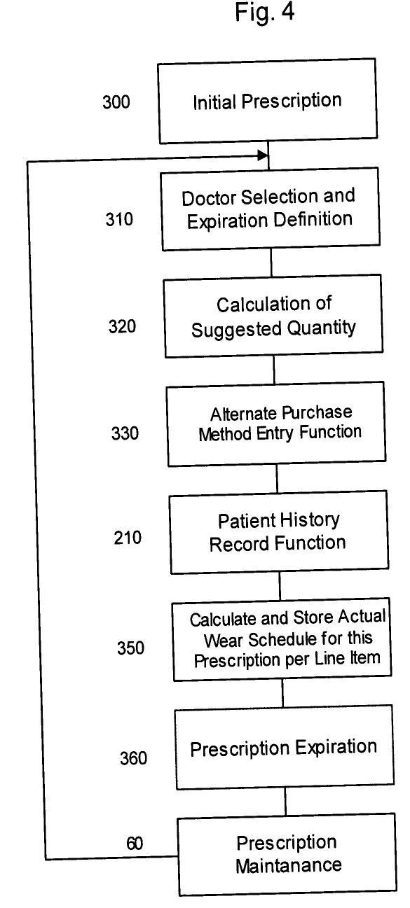

FIG. 4 is a flow diagram illustrating prescription management 30, including at least one type of prescription entry method into the initial prescription 300. In one prescription entry method the authorized reseller adds a new patient to the system, and then adds the patient's initial prescription. In a second method, the registered authorized reseller selects a patient that was previously entered using the patient management 20, and then enters the patient's initial prescription. In a third method, the consumer has completed consumer registration 40 and appears on the authorized reseller's patients awaiting prescription list. The authorized reseller may select a patient from the patients awaiting prescription list and enters an initial prescription. In this third method, the authorized reseller system 95 may send an automated notification, such as an e-mail, to the consumer informing the consumer that a prescription has been entered. The consumer can then gain authenticated access with the previously selected user id and password and place an order in the consumer system 90.

The entry of prescription details may be the same, regardless of which method the authorized reseller uses to initiate the process. The authorized reseller may select a doctor, oculist, or other similar entity in step 310 from those that have been entered in the authorized reseller online services setup 120, as the prescribing entity associated with the prescription. The authorized reseller inputs into the system the types of lenses (for example, spherical, toric, bifocal, cosmetic, combinations thereof and the like), lens parameters (diameter, power, color and the like) and the lens brand which are specified in the prescription. The authorized reseller reviews and either accepts, or modifies the default prescription period obtained from the prescription defaults entered in the authorized reseller online services setup 120. The authorized reseller may iteratively add one product at a time to the prescription. When a product is added, a suggested quantity 320 may be calculated based on the recommended wear schedule and the prescription duration. The authorized reseller reviews and either accepts or modifies the suggested quantity 320 for each item. The authorized reseller can also indicate the amount, if any, of an initial office purchase 330, wherein the patient purchased, for example, an initial supply from the authorized reseller, such as the authorized reseller's stock on hand. The consumer system 90 and the authorized reseller system 95 consider alternate purchases, such as initial office purchases, so that the patient cannot obtain more than the originally prescribed quantity by combining office and online purchases. An optional component of the prescription process is the patient history 210. Dated text descriptions of patient history 210 entered by the authorized reseller can be used to track information and activity for a patient. At the conclusion of the prescription entry process, the prescription management calculates and stores an actual wear schedule 350 for each product in the prescription. This calculation is based on the prescription duration, prescribed quantity, and number of items per box. The actual wear schedule 350 is used when determining the maximum amount that a consumer can order each time the consumer gains authenticated access to the consumer system 90 (′maximum number of purchasable boxes). The maximum number of puchasable boxes insures that at all times during the prescription duration, and particularly toward the end of the prescription duration, a consumer does not order more prescribed products than can be used in the remaining term of the prescription.

Specifically, the maximum number of purchasable boxes is calculated using the following procedure. The duration of the prescription is input by the authorized reseller along with the mode of wear (for example, daily disposable, biweekly daily wear or continuous extended wear). The specific lens product being prescribed is selected and the number of total purchasable boxes within the prescription duration is inputted. The product sales management calculates the actual wear schedule based on prescription duration and the number of purchasable boxes.

For example, when daily disposable lenses are prescribed and the number of contact lenses per box is 30, the consumer uses one contact lens in each eye each day. For a prescription with a one year duration, the consumer may purchase twelve boxes for each eye during the entire duration of the prescription. The actual wear schedule in this example is one lens per eye per day. The maximum number of purchasable boxes would be [(1× the number of days left in the prescription period)/number of lenses per box.]−number of lenses already purchased.

The authorized reseller will also select a rounding rule, to be used by the product management system in determining the number of purchasable lenses remaining when the actual wear schedule calculates a maximum number of purchasable boxes that is fractional.

The authorized reseller may select one of three rounding rules, described below.

In the first rounding rule any fractional box is rounded up to the next whole number to obtain the number of purchasable boxes. So in the example above, if a consumer were to place an order 45 days from the expiration of his/her prescription the maximum number of purchasable lenses would be 45 per eye, which is 1.5 boxes per eye. The first rounding rule would round the number of purchasable boxes for each eye up to 2 boxes per eye.

In the second rounding rule any fractional value is disregarded. Again, using the example above where a consumer places an order 45 days from the expiration of his/her prescription, the number of purchasable boxes would be 1 box per eye.

Under the third rounding rule, fractional boxes of 0.5 or more are rounded up to the nearest whole box, while fractional boxes less than 0.5 are disregarded. Thus, in the example above, the purchasable amount would be two boxes per eye. However, if the order were placed 44 days from the expiration of the prescription term, the number of purchasable boxes would be one box per eye.

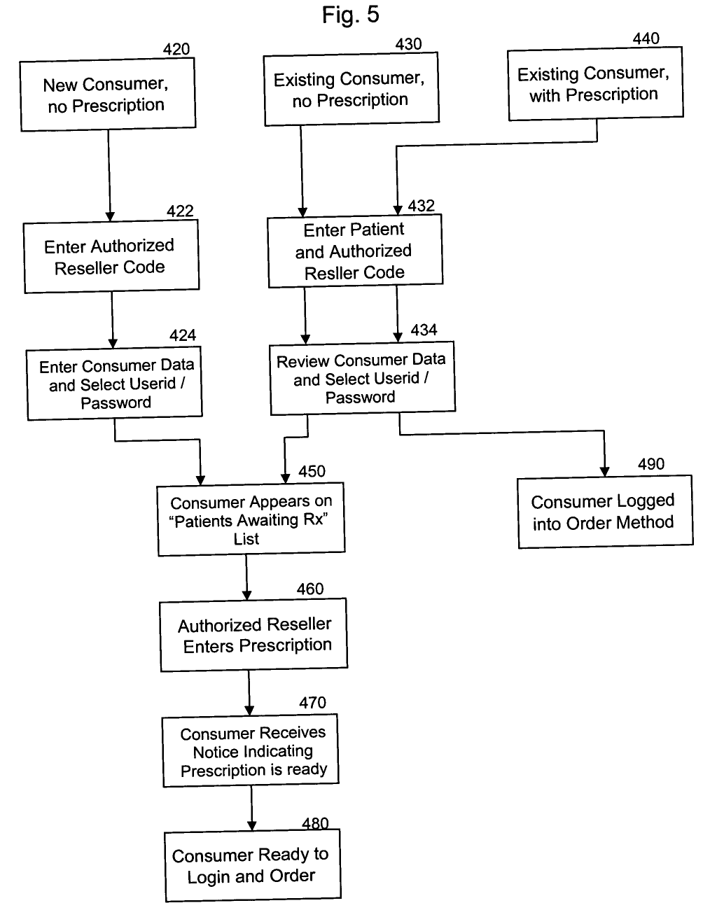

FIG. 5 is a flow diagram illustrating the consumer registration process 40. The consumer registration 40 allows the authorized reseller's consumers to register to use the consumer system 90. The registration 40 includes possible scenarios combining new and existing patients, those who have prescriptions, and those without prescriptions. To initiate the process, the patient or consumer may select the consumer registration 40 from the consumer system 90, or be sent a notice 470 that a prescription is available against which the patient can place an order.

In a first scenario 420, the authorized reseller has not yet entered a new patient's information into the authorized reseller system. The consumer accesses the consumer registration 40 and enters the authorized reseller's unique code 422 as given to the consumer by the authorized reseller, to begin consumer registration 40. The consumer enters personal information to consumer data 424, including, but not limited to, their user id and password, insurance information, mailing address, shipping address, telephone number, and e-mail address. The consumer's information is then associated with the appropriate authorized reseller. The patient then appears on the patient's awaiting prescriptions list 450 discussed hereinabove in the authorized reseller system 95 while awaiting the entering of prescription information by the authorized reseller 460. Once the authorized reseller has entered the appropriate information, the consumer receives a notice 470 indicating that the patient may place online orders.

In a second scenario 430, the authorized reseller has already entered the patient's information in the authorized reseller system 95, but the patient lacks a prescription. The consumer accesses the consumer registration 40 and enters the authorized reseller's code together with the patient code 432, both provided by the authorized reseller, reviews patient information and selects the user id and password for the online ordering system 50. As in the first scenario, the patient's information is then associated with the appropriate authorized reseller. The patient then appears on the patient's awaiting prescriptions list 450, discussed hereinabove, in the authorized reseller system 95 while awaiting the entering of prescription information by the authorized reseller 460. Once the authorized reseller has entered the appropriate information, the consumer receives a notice 470 indicating that the consumer may place online orders.

In the third scenario 440, the authorized reseller has entered the patient's information and his/her initial prescription into the authorized reseller system 95. The consumer accesses the consumer registration 40 and enters the authorized reseller's code together with the patient code 432, each of which may be provided by the authorized reseller, reviews patient information and selects the user id and password for the consumer online ordering system 50. At the completion of the registration process, a prescription may be in effect for this patient, and the consumer gains authenticated access 490 into the consumer system 90, thereby allowing the consumer to immediately place an order.

In one embodiment of the present invention one consumer or multiple consumers may be given authenticated access into the consumer system for several patients. For example, one consumer may have access and ordering authorization for several members in the consumer's family, including, for example a spouse, children, parents, combinations thereof and the like. In this way, orders for one household may be entered at one time and products may be aggregated for shipping, thereby decreasing cost and increasing convenience to the consumer.

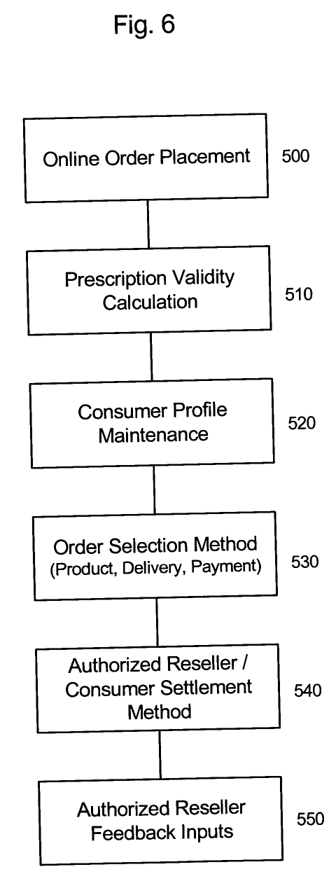

FIG. 6 is a flow diagram illustrating the consumer supply ordering process 50. The online ordering 50 allows consumers to place online orders against at least one active prescription that was entered by their respective authorized reseller. Consumers can access the online ordering 50 through a network, such as the internet or an intranet, such as via personal computer, cellular phone, or other network-capable device.

In the authentication of step 500, it is verified that the consumer has entered a valid user id and password. It is verified 510 that the consumer has either at least one active prescription with a valid order quantity greater than zero or has authority to order against at least one active prescription with a valid order quantity greater than zero for a patient. In making this determination, a calculation is performed taking into account several factors, such as the original prescription quantity, the quantity of online purchases already completed, the quantity of alternative purchases recorded by the authorized reseller, the remaining quantity that has not yet been purchased, the amount of time remaining in the prescription duration, the patient's actual wear schedule as calculated when the prescription was first entered, and the rounding rule selected by the authorized reseller in setup of authorized reseller online services 120.

Specifically, once the consumer id and password are authenticated in step 500, product sales management, 510 calculates the remaining term of the prescription period. If no term is remaining in the prescription period, the product management system notifies the consumer that the prescription has expired and a visit to an oculist and/or a new prescription is required. If there is term remaining in the prescription period the product management system calculates the number of boxes purchased by the consumer and subtracts the number of boxes purchased from the number of purchasable boxes. If the maximum purchasable boxes have already been purchased, the consumer is notified that the quantity of lenses specified in the prescription has been exceeded and a new prescription is required. After re-examination by the oculist, the authorized reseller may perform the renewal of the prescription information into the sales system.

If the number of purchasable boxes has not been exceeded the product management system calculates the maximum number of purchasable boxes, as described above, and limits the number of lenses which may be ordered to the number of lenses which could be worn according to the actual wear schedule, using the rounding rule selected by the authorized reseller during authorized reseller set up.

Accordingly, the sales system of the present invention manages the number of purchasable boxes using the maximum number of purchasable boxes and the rounding rule selected by the authorized reseller.

Thus, the sales system of the present invention provides the consumer with the convenience of direct online ordering while insuring that the consumer may not purchase the specific products such as the contact lens beyond the term of the prescription or the amount required during the prescription period.

After the consumer has successfully gained authenticated access, the system may display various authorized reseller and/or patient information, as well as provide a view of the patient's last order, and/or the ability to change information associated with a consumer profile 520, including, but not limited to, user id and password, insurance information, mailing address, shipping address, telephone number, and email address. The system may display the patient's prescription information, together with the authorized reseller's product pricing 530 for the products contained in the prescription. The products prices may be standard prices, with or without alternate pricing, or may be seasonal campaign prices, with or without alternate pricing.

The consumer selects the desired quantity of one or more products to be ordered and proceeds to select the desired delivery and payment method 530. Points of delivery may include the patient's residence, place of work, or the patient's doctor or authorized reseller's location, or other place as designated by the consumer. The consumer then performs a final review of the order and may edit any portion of it before final submission. After the order is submitted, the system displays a confirmation page containing the consumer's order number. The settlement method 540 for online orders 50 occurs after the order has been submitted. A third party clearing house may process the various methods of consumer payment and remit payment to the authorized reseller. The manufacturer may use normal and accepted billing methods to bill the authorized reseller for products ordered by patients.

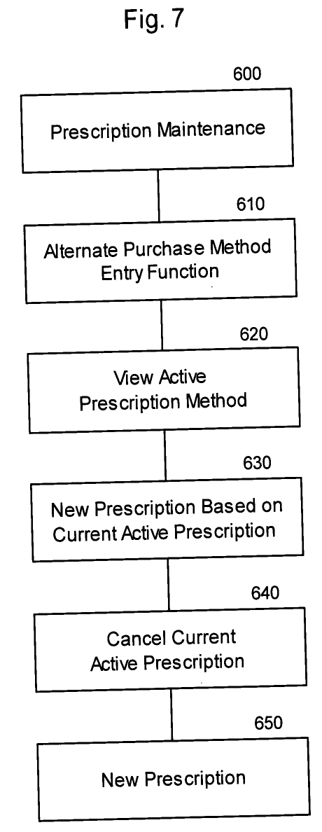

FIG. 7 is a flow diagram illustrating the prescription maintenance process 60. The prescription maintenance 60 encompasses activities that an authorized reseller can perform in relation to prescriptions after a patient's initial prescription has been entered. These activities allow the authorized reseller to manage prescriptions for their patients and ultimately control the consumer's ability to place online orders.

There are several activities that are available when the authorized reseller is viewing the active prescription 600 in the prescription maintenance 60. First, the authorized reseller can choose to record purchases made by the patient through an alternative purchase 610, such as an office purchase from the authorized reseller's on hand stock. The recording of alternative purchases 610 helps to control the total purchases made by the consumer during the prescription period. The authorized reseller can create a new prescription 630 based on a currently active prescription. This effectively cancels the current prescription and replaces it with a new one. The new prescription may contain identical products, or may be modified to meet the patient's changing requirements. The authorized reseller may cancel an active prescription 640 at any time so that the patient is no longer able to make online purchases against that prescription. The authorized reseller may begin a new prescription 650. Again, this process cancels the currently active prescription, and the authorized reseller enters the new prescription with the appropriate details for the patient. Although the authorized reseller is able to view prescription information for cancelled and expired prescriptions using the prescription maintenance 600, new prescriptions based on current active prescriptions 630 or cancellation of current prescriptions 640 may not be available when working with cancelled or expired prescription information 600.

The authorized reseller may provide additional support to the patient in conjunction with the online ordering system 50 by providing updates to the patient record, including, but not limited to, e-mail interfaces, such as automatic reminder e-mails to patients when the system calculates that the patient's supply of lenses should be getting low, e-mails to selected patients about promotions such as seasonal campaigns, and automatic reminder e-mails to patients when prescriptions are about to expire so that the patient can schedule a next appointment. Although the consumer system 90 may support automated e-mails to patients, these e-mail functions are optional. Patient e-mails may be controlled by the authorized reseller and may require the patient's prior permission.

Those skilled in the art will note, in light of the embodiments of the present invention herein, that an authorized reseller and an oculist may be a single party, may be affiliated parties, or may be separate parties. Consequently, an oculist may register separately from an authorized reseller, following a methodology similar to that disclosed hereinabove with respect to an authorized reseller, and may be invited to register in accordance with a registration of a patient, registration of an authorized reseller that inputs information of that oculist, and/or by request of the manufacturer, such as in an endeavor by that manufacturer to generate the at least one eligible prescription writer for a previously registered authorized reseller or patient. Thereby, the present invention allows for registration of at least two parties selected from a manufacturer, an authorized reseller/oculist, an oculist, and/or a patient/consumer, and allows for interactions between those at least two parties through the intermediary. Such interaction may be, for example, via automated telephone or email to one party from the intermediary in accordance with the action of at least one other party, or via invitations to register with the intermediary based on the registration, request, or prescription for, at least one other party. Thus, the present invention may seek registration of one or more of these parties in accordance with registration of another one or more of these parties.

Thus, the present invention provides a online ordering system which is managed by or on behalf of a prescribed product manufacturer. The system allows for multiple authorized resellers and oculists to have their own sites within the system, where they can set their own prices and promotions. The system also allows consumers to locate and choose between multiple oculists and authorized resellers on the system, as well as to manage a number of related patient accounts (such as family members) from one ordering page. Finally, the system of the present invention provides rigorous prescription verification, insuring that lenses cannot be purchased beyond the term of the prescription period, or that a consumer cannot order more lenses than can be used during the prescription period.

Those of ordinary skill in the art will recognize that many modifications and variations of the present invention may be implemented. The foregoing description and the following claims are intended to cover all such modifications and variations.

What is claimed is:

1. A method comprising of selling a prescribed product to a consumer via an online ordering system managed by or on behalf of a prescribed product manufacturer, comprising the steps of:
(a) registering at least one authorized reseller of at least one product;
(b) registering at least one consumer to purchase at least one product on behalf of at least on patient for whom said product has been prescribed;
(c) assessing at least one approved prescription writer for the at least one product for the at least one patient;
(d) accepting an approved prescription for the at least one product for the at least one patient from the approved prescription writer;
(e) performing at least one calculation on the approved prescription to generate a calculated approved prescription for the at least one product for the at least one patient wherein the calculated approved prescription includes a maximum amount of prescribed product that may be purchased under the calculated approved prescription and an actual wear schedule;
(f) issuing the at least one product to the consumer in accordance with the calculated approved prescription;
wherein said registering the at least one consumer is responsive to inviting of the at least one consumer by the at least one authorized reseller.

2. The method of claim 1, further comprising managing at least two prescriptions, each directly correspondent to one of at least two patients, in accordance with said performing at least one calculation.

3. The method of claim 1, wherein the at least one product comprises contact lenses.

4. The method of claim 1, further comprising managing, by the manufacturer, of said registering steps and said performing of at least one calculation.

5. The method of claim 1, wherein said issuing comprises selling the at least one product to the at least one consumer.

6. The method of claim 5, wherein said issuing further comprises delivering the at least one product to the at least one consumer.

7. The method of claim 1, wherein said issuing comprises selling the at least one product to the at least one authorized reseller, and delivering the at least one product to the at least one consumer.

8. The method of claim 7, wherein said selling to the at least one authorized reseller comprises charging a charge account dedicated to the at least one authorized reseller.

9. The method of claim 8, further comprising setting up the charge account of the charged authorized reseller during said registration of the at least one authorized reseller.

10. The method of claim 1, wherein said assessing at least one approved prescription writer comprises registering at least one doctor affiliated with at least one of the at least one authorized reseller and that cares for at least one of the at least one consumer.

11. The method of claim 1, wherein said inviting of the at least one consumer comprises:
(a) receiving a calculated approved prescription from the at least one authorized reseller;
(b) assessing that the at least one consumer related to the calculated approved prescription is not registered;
(c) receiving, from the authorized reseller, at least one contact item for the at least one consumer related to the calculated approved prescription;
(d) inviting the at least one consumer related to the calculated approved prescription to register based upon the at least one contact item.

12. The method of claim 1, wherein said inviting of the at least one consumer comprises:
(a) assessing that the at least one consumer related to the calculated approved prescription is not registered;
(b) receiving, from the authorized reseller, at least one contact item for the at least one consumer related to the calculated approved prescription;
(c) inviting the at least one consumer related to the calculated approved prescription to register based upon the at least one contact item;
(d) receiving a calculated approved prescription from the at least one authorized reseller.

13. The method of one of claims 1, 11, or 12, wherein said inviting comprises an email invitation.

14. The method of one of claims 1, 11, or 12, wherein said inviting comprises an telephonic invitation.

15. The method of claim 1 wherein the consumer may order products for multiple patients.

16. The method of claim 15 wherein the multiple products are shipped to said consumer in one shipment.

17. A system for ordering at least one prescribed product comprising:
an authorized reseller system comprising an authorized reseller registration system wherein at least one prescription is entered;
a consumer system comprising a consumer registration system and a prescribed product order placement system; and
a prescription management system comprising a prescription verification function which comprises performing at least one calculation on the at least one prescription entered by the reseller to generate a calculated approved prescription for the at least one product for the at least one consumer wherein the calculated approved prescription includes a maximum purchase amount of prescribed product that may be purchased under the calculated approved prescription and an actual wear schedule wherein said authorized reseller system, said consumer system and said prescription management system are communicatively linked so that the consumer may order prescribed products directly from a manufacturer of prescribed product so long as the amount of prescribed products ordered does not exceed the maximum purchase amount or the actual wear schedule.
